(19) United States
(12) Patent Application Publication
(10) Pub. No.: US 2003/0118647 A1
(43) Pub. Date: Jun. 26, 2003

(54) Extended release tablet of metformin

(76) Inventor: Pawan Seth, Irvine, CA (US)

Correspondence Address: HARNESS, DICKEY & PIERCE, P.L.C., P.O. BOX 828, BLOOMFIELD HILLS, MI, 48303, US

(21) Appl. No.: 10/005,387

(22) Filed: Dec. 4, 2001

(51) Int. Cl. A61K 31/155; A61K 9/22; A61K 9/32

(52) U.S. Cl. 424/468; 514/635

(57) ABSTRACT

The invention provides an extended release tablet, comprising: (i) a core comprising metformin; and (ii) a coating consisting essentially of a water-insoluble, water-permeable film-forming polymer, a plasticizer and a water-soluble polymer.

### BACKGROUND OF THE INVENTION

There is a need to obtain new release dosage form of Metformin, especially sustained or extended.

### SUMMARY OF THE INVENTION

The invention provides an extended release tablet comprising:

(i) a core comprising metformin and conventional excipients; and

(ii) a coating consisting essentially of a water-insoluble, water-permeable film-forming polymer, a plasticizer and a water-soluble polymer.

The invention thus provides a new metformin extended release composition under the form of a tablet, the core of which comprising mainly metformin. Also, the extended release is obtained thanks to a semi-permeable release coating, free of (monomeric) pore-forming agent. The tablets of the invention exhibit specific dissolution profiles.

### DETAILED DESCRIPTION OF THE INVENTION

The invention consists in a tablet comprising a core and a coating. The core includes metformin, and conventional excipients, notably a lubricant, and a binder and/or a filler, and optionally a glidant as well as other excipients.

Examples of lubricants include stearic acid, magnesium stearate, glyceryl behenate, stearyl behenate, talc, mineral oil (in PEG), sodium stearyl fumarate, etc. Glyceryl behenate is one preferred lubricant. Examples of binders include water-soluble polymer, such as modified starch, gelatin, polyvinylpyrrolidone, polyvinylalcohol (PVA), etc. The preferred binder is polyvinylalcohol. Examples of fillers include lactose, microcristalline cellulose, etc, the latter being preferred. An example of glidant is silicon dioxide (Aerosil® of Degussa). The above binders, lubricants, fillers, glidants, and any other excipient that may be present can further be found in the relevant literature, for example in the Handbook of Pharmaceutical Excipients. The relative amounts of ingredients in the core are preferably as follows. The proportion of metformin in the core may vary between 70 and 99%, preferably 85 and 98%, of the core dry weight. The proportion of lubricant and/or glidant in the core may vary between 0.3 and 10%, preferably 0.5 to 3%, of the core dry weight. The proportion of binder or filler in the core may vary between 0.5 and 25%, preferably 1 to 10%, of the core dry weight.

The core may further comprise, according to one embodiment of the invention, an expanding agent. The expanding agent will lead to an expansion of e.g. 10 to 35% vol., especially 15 to 30% vol. This expansion will allow the drug to say longer in the stomach, thus in fed conditions (metformin is generally to be taken in fed conditions, since the metformin absorption mechanism is considered to be mainly through the intestine walls). An example of expanding agent is Na starch glycolate (Primogel®); any agent that swells with water can be used, e.g. known desintegrant agents. The proportion of expanding agent, when one is present, in the core may vary between 3 and 25%, preferably 5 to 20%, of the core dry weight.

The manufacturing process of the core can be as follows. Metformin is first granulated with a binder, in a granulator, preferably but not necessarily a fluidized bed granulator. The binder is first dissolved or dispersed in a suitable solvent, preferably water. The solution or suspension of binder is then sprayed onto the drug in a granulator, e.g. fluidized bed granulator. For example, fluidized bed granulators manufactured by Glatt (Germany) or Aeromatic (Switzerland) can be used for this operation. An alternative process can be to use a conventional or high shear mixer to proceed granulation. If necessary, the drug can be mixed with a filler, prior to the granulation step. Granules once dried can be mixed with the other excipients, especially with the lubricant and the expanding agent if present, but also with glidants and any other excipient suitable to improve processing. The mixture of granules (preferably with lubricant), and optionally glidant is pressed into tablets. Alternatively, the active ingredient and lubricant and/or glidant and/or expanding agent can be mixed in a granulator, e.g. a fluidized bed granulator, and heated to the melting point of the lubricant to form granules. This mixture can then be mixed with a suitable filler and compressed into tablets (the expanding agent may also be added at that stage). Also, it is possible to mix the active ingredient and the lubricant (e.g. glyceryl behenate) and the expanding agent if present in a granulator, e.g. a fluidized bed granulator, and then to press the resulting granules into tablets. Tablets can be obtained by standard techniques, e.g. on a (rotary) press (for example Manesty Betapress®) fitted with suitable punches. The resulting tablets are hereinafter referred as tablet cores.

These tablet cores are then coated with the semi-permeable coating designed to achieve an extended release of metformin. The coating comprises a water-insoluble, water-permeable film-forming polymer, together with a plasticizer and a water-soluble polymer.

The water-insoluble, water-permeable film-forming polymer can be a cellulose ether, such as ethylcellulose, a cellulose ester, such as cellulose acetate, etc. The preferred film-forming polymer is ethylcellulose (available from Dow Chemical under the trade name Ethocel®). The plasticizer can be an ester such as a citrate ester or dibutyl sebacate, an oil such as castor oil, a polyalkyleneglycol such as polyethyleneglycol of various MWs, a fatty acid such as stearic acid. The preferred plasticizer are dibutyl sebacate and stearic acid. The water-soluble polymer is preferably polyvinylpyrrolidone. Some other excipients can be used in the coating, as for example acrylic acid derivatives (available from Roehm Pharma under the trade name “Eudragit®”), pigments, etc. The relative amounts of ingredients in the coating are preferably as follows. The proportion of water-insoluble, water-permeable polymer (e.g. ethylcellulose) in the coating may vary between 20 and 85% of the coating dry weight. The proportion of water-soluble polymer (e.g. polyvinylpyrrolidone) in the coating may vary between 10 and 75% of the coating dry weight. The proportion of plasticizer (e.g. stearic acid) in the coating may vary between 3 and 40% of the coating dry weight. The relative proportions of ingredients, notably the ratio water-insoluble, water-permeable film-forming polymer to water-soluble polymer and to plasticizer, can be varied depending on the release profile to be obtained (where a more extended release is generally obtained with a higher amount of water-insoluble, water-permeable film-forming polymer) and depending on the presence of an expanding agent in the core (which usually leads to more plasticizer and less water-insoluble, water-permeable film-forming polymer).

For example, the following are preferred proportions water-insoluble, water-permeable film-forming polymer/water-soluble polymer/plasticizer:

Without any expanding agent: 50-85/10-35/3-15;

With an expanding agent: 20-50/35-75/15-40.

The coating process can be as follows. Ethylcellulose, dibutyl sebacate (or stearic acid) and polyvinylpyrrolidone are dissolved in a solvent such as ethanol. The resulting solution is sprayed onto the tablet cores, using a coating pan or a fluidized bed apparatus. The weight ratio coating/tablet core is comprised e.g. between 1/50 and 5/10, preferably between 2/100 and 20/100, e.g. from 5/100 to 10/100.

The tablet comprises an amount of metformin that can vary within broad limits, such as from 400 to 2000 mg. For example, this amount can be from 550 mg to 2000 mg per tablet, with exemplary ranges being: 600-1800 mg; 700-1500 mg; 800-1300 mg; 900-1100 mg; especially about 1000 mg. For example, this amount can be from 400 to 550 mg; especially about 500 mg. Surprisingly, it was discovered that the above formulation did not lead to any degradation of metformin though no stabilizer was present in the formulation. Stability studies were conducted in oven, under the storage test conditions described in the US pharmacopoeia 23^{rd} edition page 1961. Under these conditions no significant change in drug potency could be seen. Surprisingly, it was also discovered that the above formulation did provide an extended (sustained) release though no pore-forming agent was present in the coating.

The invention thus provides a metformin extended release tablet free of stabilizer and free of pore-forming agent, exhibiting a dissolution profile such that after 2 hours, from 7 to 60% of the metformin is released; after 4 hours, from 15 to 90% of the metformin is released; after 8 hours, from 50 to 100% of the metformin is released; after 12 hours, more than 75% of the metformin is released.

According to one embodiment, the dissolution profile is such that after 2 hours, from 10 to 40% of the metformin is released; after 4 hours, from 20 to 65% of the metformin is released; after 8 hours, from 50 to 100% of the metformin is released; after 12 hours, more than 75% of the metformin is released. According to another embodiment, the dissolution profile is such that after 2 hours, from 40 to 60% of the metformin is released; after 4 hours, from 65 to 90% of the metformin is released; after 8 hours, from 85 to 100% of the metformin is released; after 12 hours, more than 90% of the metformin is released.

### BEST MODES FOR CARRYING THE INVENTION

A preferred tablet composition comprises:

(i) a core comprised of metformin, polyvinylalcohol, silicon dioxide and glyceryl behenate; and

(ii) a coating comprised of ethylcellulose, polyvinylpyrrolidone and stearic acid or dibutyl sebacate.

Another preferred tablet composition is one in which the core additionally comprises an expanding agent, preferably Na starch glycolate.

### EXAMPLES

The following examples illustrate the invention without limiting it. The amounts are given per dosage form.

### Example 1A

The following formulation is prepared.

| Ingredients | Amount (mg) |
|---|---|
| Metformin | 1000.00 |
| Polyvinylalcohol PVAe | 25.00 |
| Silicon dioxide | 20.00 |
| Glyceryl behenate | 21.00 |
| Total (dry weight) | 1066.00 |

Metformin and silicon dioxide are placed in a fluidized bed apparatus. An aqueous PVA solution (at 1% by weight) is sprayed to get granules. The apparatus is a Glatt GPCG1, operated with the following parameters.

| | | |
|---|---|---|
| Air flow (m³/h) | 100-110 | m³/h |
| Liquid flow (g/min) | 6-7 | g/min |
| Inlet temperature | 65° | C. |
| Spraying pressure | 2.8 | bar |

The granules thus obtained are subsequently dried. Then they are passed through a sieve (1 mm mesh) and glyceryl behenate is weighed, added and blended in a drum mixer (Turbula T2C, Bachoffen, Switzerland). The resulting mixture is pressed into tablets (7 mm diameter and 7 mm curvature) with average hardness being between 60 and 120N. These tablet cores are then coated with the following formulation.

| Ingredients | Amount (mg) |
|---|---|
| Tablet cores | 1066.00 |
| Ethocel PR100 (ethylcellulose) | 42.63 |
| Kollidon 90F (povidone USP) | 14.98 |
| Stearic acid | 6.39 |
| Total (dry weight) | 1130.00 |

Ethocel, povidone and stearic acid are first dissolved in denatured alcohol (550 g). The coating solution is then sprayed onto the tablet cores in a coating pan (Vector LCDS), with the following spraying parameters:

| | | |
|---|---|---|
| Air flow (m³/h) | 100-110 | m³/h |
| Liquid flow (g/min) | 6-7 | g/min |
| Inlet temperature | 65° | C. |
| Spraying pressure | 2.8 | bar |

Stability Data:

Storage conditions: conforms to U.S. Pat. No. 23 guideline (25° C. and 60% relative humidity and 40° C. and 75% relative humidity). The results show that the Metformin composition of this example is stable.

### Example 1B

Example 1A is reproduced (same manufacture process), with the following formulation for the core.

| Ingredients | Amount (mg) |
|---|---|
| Metformin | 500.00 |
| Polyvinylalcohol PVAe | 12.50 |
| Silicon dioxide | 10.00 |
| Glyceryl behenate | 10.50 |
| Total (dry weight) | 533.00 |

The coating has the following formulation.

| Ingredients | Amount (mg) |
|---|---|
| Tablet cores | 533.00 |
| Ethocel PR100 (ethylcellulose) | 26.50 |
| Kollidon 90F (povidone USP) | 9.55 |
| Stearic acid | 3.95 |
| Total (dry weight) | 573.00 |

The stability results show that the Metformin composition of this example is stable.

### Example 2A

Example 1A is reproduced, but with the following coating formulation.

| Ingredients | Amount (mg) |
|---|---|
| Tablet cores | 1066.00 |
| Ethocel PR100 (ethylcellulose) | 41.33 |
| Kollidon 90F (povidone USP) | 16.47 |
| Stearic acid | 6.20 |
| Total (dry weight) | 1130.00 |

The stability results show that the Metformin composition of this example is stable.

### Example 2B

Example 1B is reproduced, but with the following coating formulation:

| Ingredients | Amount (mg) |
|---|---|
| Tablet cores | 533.00 |
| Ethocel PR100 (ethylcellulose) | 25.24 |
| Kollidon 90F (povidone USP) | 7.97 |
| Stearic acid | 3.79 |
| Total (dry weight) | 570.00 |

The stability results show that the Metformin composition of this example is stable.

### Example 3A

The following formulation is prepared.

| Ingredients | Amount (mg) |
|---|---|
| Metformin | 1000.00 |
| Polyvinylalcohol PVAe | 25.00 |
| Silicon dioxide | 25.00 |
| Glyceryl behenate | 23.00 |
| Primogel NF 17 | 100.00 |
| Total (dry weight) | 1173.00 |

The same procedure as in example 1A is followed, except that Primogel® is added at the same time as glyceryl behenate.

The tablet cores thus-obtained are then coated with the following formulation.

| Ingredients | Amount (mg) |
|---|---|
| Tablet cores | 1173.00 |
| Ethocel PR100 (ethylcellulose) | 30.60 |
| Kollidon 90F (povidone USP) | 37.40 |
| Dibutyl sebacate | 17.00 |
| Total (dry weight) | 1258.00 |

The same procedure as in example 1A is followed, except that stearic acid is replaced by dibutyl sebacate. The stability results show that the Metformin composition of this example is stable.

### Example 3B

Example 3A is reproduced (same manufacture process), with the following formulation for the core.

| Ingredients | Amount (mg) |
|---|---|
| Metformin | 500.00 |
| Polyvinylalcohol PVAe | 12.50 |
| Silicon dioxide | 10.00 |
| Glyceryl behenate | 10.50 |
| Primogel NF 17 | 50.00 |
| Total (dry weight) | 533.00 |

The coating has the following formulation.

| Ingredients | Amount (mg) |
|---|---|
| Tablet cores | 583.00 |
| Ethocel PR100 (ethylcellulose) | 21.25 |
| Kollidon 90F (povidone USP) | 21.25 |
| Dibutyl sebacate | 10.60 |
| Total (dry weight) | 636.10 |

The stability results show that the Metformin composition of this example is stable.

### Example 4

### Dissolution Profiles

The dissolution profile is determined in the following dissolution conditions:

Medium: 900 ml phosphate buffer pH 6.8.

Method: 75 rpm USP Apparatus I.

The results are given in % in the following table.

Time (hour)

| | 2 | 4 | 8 | 12 |
|---|---|---|---|---|
| Example 1A | 13.8 | 32.9 | 69.3 | 91.9 |
| Example 1B | 23.8 | 53.2 | 92.3 | 100.0 |
| Example 2A | 23.8 | 51.0 | 89.4 | 100.0 |
| Example 2B | 11.5 | 29.1 | 67.3 | 92.3 |
| Example 3A | 29.1 | 51.9 | 80.2 | 91.8 |
| Example 3B | 53.6 | 84.6 | 100.0 | N/A |

The invention is not limited to the specific embodiments described above but can be varied within broad limits by the skilled man.

The invention claimed is:

1. An extended release tablet comprising:
(i) a core comprising by weight, based on the core weight, 70 to 99% of metformin, and conventional excipients; and
(ii) a coating consisting essentially by weight, based on the coating weight, of 20 to 85% of a water-insoluble, water-permeable film-forming polymer, of 10 to 75% of a water-soluble polymer and 3 to 40% of a plasticizer,
exhibiting a dissolution profile such that after 2 hours, from 7 to 60% of the metformin is released; after 4 hours, from 15 to 90% of the metformin is released; after 8 hours, from 50 to 100% of the metformin is released; after 12 hours, more than 75% of the metformin is released.

2. The tablet of claim 1, where the water-insoluble, water-permeable film-forming polymer is ethylcellulose.

3. The tablet of claim 1, where the water-soluble polymer is polyvinylpyrrolidone.

4. The tablet of claim 1, where the plasticizer is stearic acid.

5. The tablet of claim 1, where the plasticizer is dibutyl sebacate.

6. The tablet of claim 1, where the water-insoluble, water-permeable film-forming polymer is ethylcellulose, the water-soluble polymer is polyvinylpyrrolidone and the plasticizer is stearic acid.

7. The tablet of claim 1, where the water-insoluble, water-permeable film-forming polymer is ethylcellulose, the water-soluble polymer is polyvinylpyrrolidone and the plasticizer is dibutyl sebacate.

8. The tablet of claim 1, where the weight proportions water-insoluble, water-permeable film-forming polymer/water-soluble polymer/plasticizer are 50-85/10-35/3-15.

9. The tablet of claim 1, where the core further comprises an expanding agent, in an amount of 3 to 25%, of the core dry weight.

10. The tablet of claim 9, where the expanding agent is Na starch glycolate.

11. The tablet of claim 9, where the weight proportions water-insoluble, water-permeable film-forming polymer/water-soluble polymer/plasticizer are 20-50/35-75/15-40.

12. The tablet of claim 1, where the core comprises glyceryl behenate, polyvinylalcohol and silicon dioxide.

13. The tablet of claim 1, exhibiting a dissolution profile such that after 2 hours, from 10 to 40% of the metformin is released; after 4 hours, from 20 to 65% of the metformin is released; after 8 hours, from 50 to 100% of the metformin is released; after 12 hours, more than 75% of the metformin is released.

14. The tablet of claim 1, exhibiting a dissolution profile such that after 2 hours, from 40 to 60% of the metformin is released; after 4 hours, from 65 to 90% of the metformin is released; after 8 hours, from 85 to 100% of the metformin is released; after 12 hours, more than 90% of the metformin is released.

15. An extended release tablet comprising:
(i) a core comprising by weight, based on the core weight, 70 to 99% of metformin, and conventional excipients; and
(ii) a coating consisting essentially by weight, based on the coating weight, of 50 to 85% of a water-insoluble, water-permeable film-forming polymer, of 10 to 35% of a water-soluble polymer and 3 to 15% of a plasticizer;
exhibiting a dissolution profile such that after 2 hours, from 7 to 60% of the metformin is released; after 4 hours, from 15 to 90% of the metformin is released; after 8 hours, from 50 to 100% of the metformin is released; after 12 hours, more than 75% of the metformin is released.

16. The tablet of claim 15, where the water-insoluble, water-permeable film-forming polymer is ethylcellulose, the water-soluble polymer is polyvinylpyrrolidone and the plasticizer is stearic acid.

17. The tablet of claim 15, where the core comprises glyceryl behenate, polyvinylalcohol and silicon dioxide.

18. The tablet of claim 15, exhibiting a dissolution profile such that after 2 hours, from 10 to 40% of the metformin is released; after 4 hours, from 20 to 65% of the metformin is released; after 8 hours, from 50 to 100% of the metformin is released; after 12 hours, more than 75% of the metformin is released.

19. The tablet of claim 15, exhibiting a dissolution profile such that after 2 hours, from 40 to 60% of the metformin is released; after 4 hours, from 65 to 90% of the metformin is released; after 8 hours, from 85 to 100% of the metformin is released; after 12 hours, more than 90% of the metformin is released.

20. An extended release tablet comprising:
(i) a core comprising by weight, based on the core weight, 70 to 99% of metformin, an expanding agent and conventional excipients; and
(ii) a coating consisting essentially by weight, based on the coating weight, of 20 to 50% of a water-insoluble, water-permeable film-forming polymer, of 35 to 75% of a water-soluble polymer and 15 to 40% of a plasticizer;
exhibiting a dissolution profile such that after 2 hours, from 7 to 60% of the metformin is released; after 4 hours, from 15 to 90% of the metformin is released; after 8 hours, from 50 to 100% of the metformin is released; after 12 hours, more than 75% of the metformin is released.

21. The tablet of claim 20, where the water-insoluble, water-permeable film-forming polymer is ethylcellulose, the water-soluble polymer is polyvinylpyrrolidone and the plasticizer is dibutyl sebacate.

22. The tablet of claim 20, where the expanding agent represents 3 to 25% of the core dry weight, and is Na starch glycolate.

23. The tablet of claim 20, where the core comprises glyceryl behenate, polyvinylalcohol and silicon dioxide.

24. The tablet of claim 20, exhibiting a dissolution profile such that after 2 hours, from 10 to 40% of the metformin is released; after 4 hours, from 20 to 65% of the metformin is released; after 8 hours, from 50 to 100% of the metformin is released; after 12 hours, more than 75% of the metformin is released.

25. The tablet of claim 20, exhibiting a dissolution profile such that after 2 hours, from 40 to 60% of the metformin is released; after 4 hours, from 65 to 90% of the metformin is released; after 8 hours, from 85 to 100% of the metformin is released; after 12 hours, more than 90% of the metformin is released.

26. The tablet of claim 1, comprising from 400 to 2000 mg metformin.

27. The tablet of claim 26, comprising from 550 to 2000 mg metformin.

28. The tablet of claim 13, comprising from 550 to 2000 mg metformin.

29. The tablet of claim 14, comprising from 550 to 2000 mg metformin.

30. The tablet of claim 26, comprising from 400 to 550 mg metformin.

31. The tablet of claim 13, comprising from 400 to 550 mg metformin.

32. The tablet of claim 14, comprising from 400 to 550 mg metformin.

33. The tablet of claim 15, comprising from 400 to 2000 mg metformin.

34. The tablet of claim 33, comprising from 550 to 2000 mg metformin.

35. The tablet of claim 18, comprising from 550 to 2000 mg metformin.

36. The tablet of claim 19, comprising from 550 to 2000 mg metformin.

37. The tablet of claim 33, comprising from 400 to 550 mg metformin.

38. The tablet of claim 18, comprising from 400 to 550 mg metformin.

39. The tablet of claim 19, comprising from 400 to 550 mg metformin.

40. The tablet of claim 20, comprising from 400 to 2000 mg metformin.

41. The tablet of claim 40, comprising from 550 to 2000 mg metformin.

42. The tablet of claim 24, comprising from 550 to 2000 mg metformin.

43. The tablet of claim 25, comprising from 550 to 2000 mg metformin.

44. The tablet of claim 40, comprising from 400 to 550 mg metformin.

45. The tablet of claim 24, comprising from 400 to 550 mg metformin.

46. The tablet of claim 25, comprising from 400 to 550 mg metformin.
